(19) United States
(12) Patent Application Publication
(10) Pub. No.: US 2015/0246183 A1
(43) Pub. Date: Sep. 3, 2015

(54) NEEDLE-FREE INJECTION DEVICES, SYSTEMS AND METHODS

(71) Applicant: Aleksandr A. Kavokin, Moscow (RU)

(72) Inventor: Aleksandr A. Kavokin, Moscow (RU)

(73) Assignee: Biopreme Medical Technologies ,Inc. a corporation

(21) Appl. No.: 14/437,167

(22) PCT Filed: Oct. 18, 2013
(86) PCT No.: PCT/US2013/065778

§ 371 Date: Apr. 20, 2015

Related U.S. Application Data
(60) Provisional application No. 61/716,497, filed on Oct. 20, 2012.

(51) Int. Cl. A61M 5/32

(52) U.S. Cl. CPC A61M 5/3286; A61M 2250/00

(57) ABSTRACT

An improved needle-free device for injecting or aspirating fluids using manually or mechanically generated injection is disclosed. Also disclosed are systems and methods for using the device, including systems having fluid reservoirs and simple negative pressure mechanisms as well as methods for using them. The devices, systems and methods disclosed herein are easy to fabricate and can deliver variable doses in a simple, effective, patient-friendly, cost-effective and disposable manner.

### CROSS-REFERENCE TO RELATED APPLICATION

This application claims the benefit of U.S. Provisional Application No. 61/716,497 filed Oct. 20, 2012.

### BACKGROUND

### State of the Art

1. Technical Field

This invention relates generally to injection or aspiration devices, systems and methods and, more particularly, to needle-less injection or aspiration devices. The devices, systems and methods may be used for medical applications as well as in any other field requiring injection including without limitation veterinary, food processing, and any production that requires injection of fluid into a substance.

2. State of the Art

Subcutaneous, intramuscular and other modes of delivery for medicates by injection in the medical arts are typically accomplished via a needle that punctures the skin of the patient. Many medical conditions require frequent daily injections including diabetes, HIV, hepatitis, allergic reactions and multiple sclerosis. Furthermore, in circumstances that require inoculations of a large number of persons, such as is often the case in the military services, the inoculations should ideally be accomplished quickly and easily.

Even with the availability of fine gauge needles, a common problem is that many people dislike needle injections due to pain, fear and nervousness over needles. In addition, the use of needles for injections can present serious risks to medical workers due to accidental needle-sticks and the possible transmission of blood-borne pathogens such as HIV and hepatitis. Specific environments such as emergency rooms, county hospitals, EMT response sites and mass immunization locations account for hundreds of thousands of accidental needle sticks annually. The consequence is billions of dollars in annual costs associated with testing, treatment of medical complications and other related costs. Furthermore, regulatory requirements regarding disposal of biohazard sharps also generate costs. Therefore, primarily for these reasons, there is a real need for needle-free injection systems.

Information relevant to attempts to alleviate such problems by using needle-free devices, methods or systems can be found in the following references: U.S. Pat. Nos. 7,284,477, 4,913,699, 5,503,627, 4,722,729, 6,447,475, 2,743,723, 5,911,703, 6,716,190, 0,210,188, and 4,403,609. Needle-free devices, methods and systems can be advantageous in that, because there is no needle, they typically do not cause much fear in patients. Needle-free devices or systems can also be used by lesser-trained individuals if necessary such as, for example, in a mass vaccination setting. In current technologies, a needle-free injection system typically incorporates a device that injects fluid via a high-pressure jet having a relatively small diameter through the skin. However, these and the other types of needle-free devices disclosed in the references above suffers from one or more of the following disadvantages:
1. the device is heavy or otherwise difficult to manipulate or use;
2. The design is complicated or otherwise difficult to fabricate or use;
3. bruising or lacerations are produced;
4. imprecise infusions of the medicates or other fluids are injected;
5. complicated steps are required to assemble and load drugs or other fluids prior to injection;
6. the units are non-disposable
7. The units require sterilization or additional precautions to prevent the transfer of contamination from one patient to another;
8. they are costly to fabricate or use;
9. they are relatively large or otherwise hard to manipulate; and
10. The units can only be used with pre-packaged doses of drugs or other fluids.

Other approaches to improve needle-less systems have recently been developed that alleviate some of the problems outlined above. Such systems may be disposable, smaller and less costly than the older systems, for example. However, these improvements have several practical disadvantages, the most significant being complexity of design and the ability to only use prepackaged doses of drugs.

Thus there remains a need for simple, easy to use, needle-free fluid or drug delivery devices, methods and systems that can inject variable dosages in a more patient-friendly, cost effective manner and which are disposable.

### BRIEF DESCRIPTION OF THE DRAWINGS

Further objects, features and advantages of the invention will become apparent from the detailed description, below, when read in conjunction with the accompanying drawings in which:

FIG. 1 is an elevation view of one embodiment of a fluid injection device having a flexible or resilient contact member;

FIG. 2 is an elevation view of one embodiment of a fluid injection device having a rigid or semi-rigid contact member and a bulb for generating a negative pressure or vacuum in the empty space between the contact member and a surface;

FIG. 3. is a plan view of a part of a method of injection using the device illustrated in FIG. 1;

FIG. 4 is a plan view of another part of a method of injection using the device illustrated in FIG. 1; and

FIG. 5 is a plan view of yet another part of a method of injection using the device illustrated in FIG. 1.

### DETAILED DESCRIPTION

The following description is of a best mode presently contemplated for practicing the invention. This description is not to be taken in a limiting sense but is made merely for the purpose of describing the general principles of the invention whose scope may be ascertained by referring to the appended claims.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of the “a” or “an” are employed to describe elements and components of the invention. This is done merely for convenience and to give a general sense of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although a few suitable, exemplary processes and materials are described below, other processes and materials similar or equivalent to those described herein can also be used in the practice or testing of the invention. All publications, patent applications, patents, and other references mentioned herein are incorporated by reference in their entirety. In case of conflict, the present specification, including definitions, will control. In addition, the materials, processes, and examples are illustrative only and not intended to be limiting.

The following definitions refer to the particular embodiments described herein and are not to be taken as limiting; the invention includes equivalents for other undescribed embodiments.

As used herein, the term “fluid” is intended to mean a substance that has no fixed shape and yields easily to external pressure; a gas or (especially) a liquid.

As used herein, the term “needle” is intended to mean a relatively thin, pointed steel tube that can be pushed through a surface such that fluids or a gas can be injected into, or removed from, a location within or below the surface.

As used herein, the term “syringe” is intended to mean a device having a hollow tube or barrel, fitted with a plunger and a hollow needle, which can be used to force fluids into, or take fluids out of, a location within or below a surface such as skin.

As used herein, the term “needle-free syringe” is intended to mean a syringe that is not fitted with a hollow needle.

The invention disclosed herein relates generally to injection and aspiration devices, systems and methods and is particularly directed to improved needle-free (needleless) injection or aspiration devices, methods and systems for delivering fluids to, or aspirating fluids from, subjects such as humans or animals in a simple, relatively painless and low cost manner. The device may be used in medicine as well as in any other field requiring an injection, including without limitation veterinary, food processing, and any fabrication or process that requires injection of a fluid into a substance.

In one embodiment, the invention can provide a needle-free injector device including a support member having an entry port for supporting a reservoir and fluidly connecting the reservoir to the device, a channel member having a fluidly connected input port and injection port, where the input port is also fluidly connected to the entry port. The injection port in this embodiment can open into an empty space defined by a overlying contact member that can be placed adjacent to a surface to be injected or aspirated, such as skin.

In another embodiment, the device can also include a negative pressure mechanism capable of creating a negative pressure or a vacuum underlying the contact member in the device. The contact member in this embodiment can include an additional port to which the negative pressure mechanism can be attached.

In another embodiment, a system according to the invention can include the device and a needle-free syringe. In yet another embodiment, a system can further include a surface or subject suitable for injection or aspiration, including without limitation synthetic or living tissue such as skin.

There are also methods for using the device or system. In one embodiment using a flexible or resilient suction cup as the contact member, the process of injection can be as follows:

(a) a syringe is filled with a fluid;

(b) the syringe is attached to the device;

(c) the integral unit (the device and syringe), loaded with a selected dosage of fluid, can be grasped in the hand of a user (or any other suitable mechanism for holding the unit), and held proximate to the epidermis in order to prepare to manually or mechanically inject the selected dosage into, through or under the epidermis;

(d) a negative pressure required for injection can be created under the suction cup. This can, for example, be done by applying pressure to the whole unit against the epidermis and, as a result, pushing all or substantially all of the air out from under the suction cup. The negative pressure thus created can draw the epidermis and, if necessary, the underlying tissue or tissues towards the injection port, creating a negative pressure in the epidermis or the tissue(s) that can facilitate piercing of the epidermis by the now adjacent injection port (including without limitation an injection port having a short protrusion); and

(e) the user can then push down on the syringe plunger, driving a piston in the plunger that ejects the selected dosage of medication from the syringe, through the injection port(including without limitation a narrow orifice of the device) into the epidermis and into or under the epidermis or tissue(s). In, this manner, this embodiment provides the same result as that achieved during typical injection with a syringe needle but without the complications inherent in using a needle or high pressure jet apparatus. A similar but reverse series of steps could similarly be used to aspirate liquid from beneath a surface.

In another embodiment using a semi-rigid or rigid suction cup as the contact member and a resilient or flexible bulb for generating a negative pressure or vacuum, the process of injection can be as follows:

(a) a syringe is filled with a fluid;

(b) the syringe is attached to a syringe hub on the device;

(c) the integral unit (the device and syringe), loaded with a selected dosage of fluid, can be grasped in the hand of a user (or any other suitable mechanism for holding the unit), and held proximate to the epidermis in order to prepare to manually or mechanically inject the selected dosage into, through or under the epidermis;

(d) the negative pressure or vacuum required for injection can be created under the suction cup. This can, for example, be done by squeezing the bulb to force substantially all or all of the air out of the bulb, placing the suction cup adjacent to the epidermis, and releasing the pressure on the bulb, thereby pulling all or substantially all of the air out from under the suction cup and into the bulb. The negative pressure thus created can draw the epidermis or underlying tissue or tissues towards the injection port, creating negative pressure in the epidermis or the tissue that can facilitate the piercing of the epidermis by the now adjacent injection port (including without limitation an injection port having a short protrusion); and

(e) the user can then push down on the syringe plunger, driving a piston in the plunger that ejects the selected dosage of medication from the syringe, through the injection port(including without limitation a narrow orifice of the device) and into, through or under the epidermis). In this manner, this embodiment provides the same result as that achieved during typical injection with a syringe needle but without the complications inherent in using a needle or high pressure jet apparatus. A similar but reverse series of steps could similarly be used to aspirate liquid from beneath a surface.

In yet another embodiment, a negative pressure or vacuum required for injection can be created under the suction cup by using a reverse piston instead of a bulb.

### EXAMPLE 1

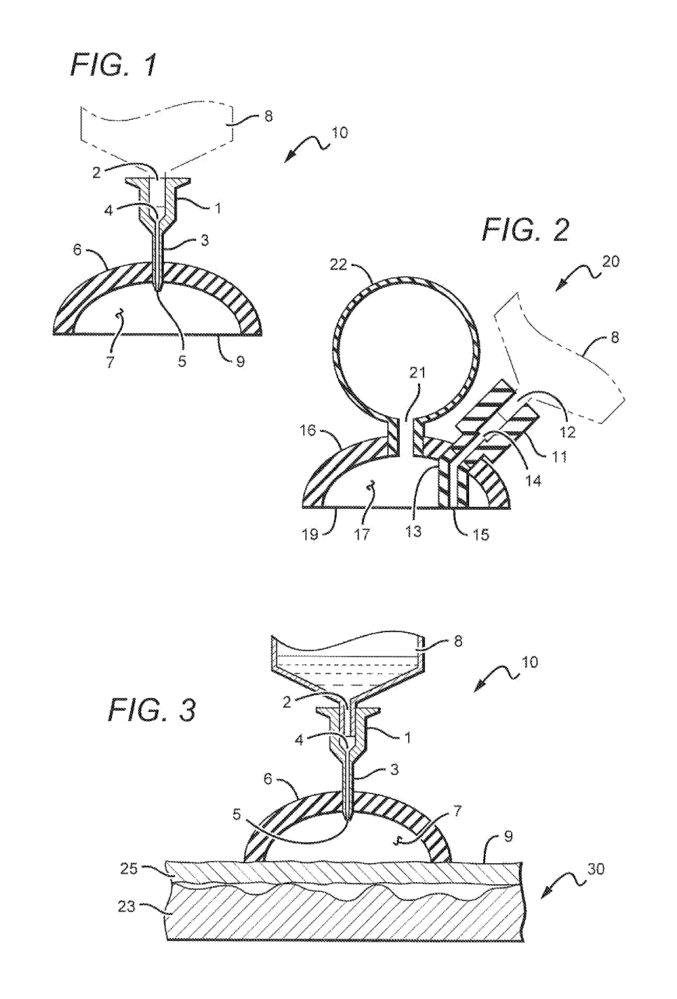

A needle-free injection or aspiration device 10 can include a syringe holder (hub) 1 having an opening for a syringe tip 2, a duct 3 having a nozzle head with a fluid input port 4 and a distinct injection port 5 such as a narrow orifice, and a suction cup 6 as illustrated in FIG. 1. The injection port 5 in this embodiment can open into the suction cup 6 and the duct 3 has a through channel that fluidly connects the input port 4 and the injection port 5. The empty space 7 in this device, which is defined by the size, shape, or dimensions of the overlying suction cup 6, can be quickly, easily and accurately placed adjacent to a surface 9 to be injected or aspirated, such as epidermis.

### EXAMPLE 2

In this embodiment, a needle-free injection or aspiration device 30 can include a syringe holder (hub) 11 having an opening for a syringe tip 12, a duct 13 having a nozzle head with a fluid input port 14 and a distinct narrow orifice injection port 15, and a suction cup 16 as illustrated in FIG. 2. The injection port 15 in this embodiment can open into the suction cup 16 and the duct 13 has a through channel that fluidly connects the input port 14 and the injection port 15. This embodiment further includes a negative pressure mechanism 22, such as a resilient or flexible bulb 22 for providing a negative pressure or vacuum in the suction cup 16 and attached to the suction cup 16 via a second input port or pressure port 21 on the suction cup 16. The narrow orifice injection port 15 and bulb 22 thus both open into the suction cup 16 in this embodiment. The bulb can be used to create a negative pressure or vacuum in the suction cup 16. The empty space 17 in this device, which is defined by the size, shape, or dimensions of an overlying flexible or resilient suction cup 16, can be quickly, easily and accurately placed adjacent to a surface 19 to be injected or aspirated, such as epidermis. This embodiment can have a suction cup 16 made at least in part of relatively rigid or hard materials because the negative pressure or vacuum in this embodiment can be created by a negative pressure mechanism (bulb) 22 rather than by deforming the suction cup 16 by hand as for the embodiment disclosed in Example 1.

### EXAMPLE 3

In this embodiment, the bulb 22 as illustrated in FIG. 2, is replaced with a reverse piston (not shown) for creating a negative pressure or vacuum, or in some embodiments, the reverse piston may be used in addition to the bulb 22. This embodiment can have a suction cup 16 made at least in part of relatively rigid or hard materials because the negative pressure or vacuum in this embodiment can be created by a negative pressure mechanism rather than by deforming the suction cup 16 by hand as for the embodiment disclosed in Example 1.

### EXAMPLE 4

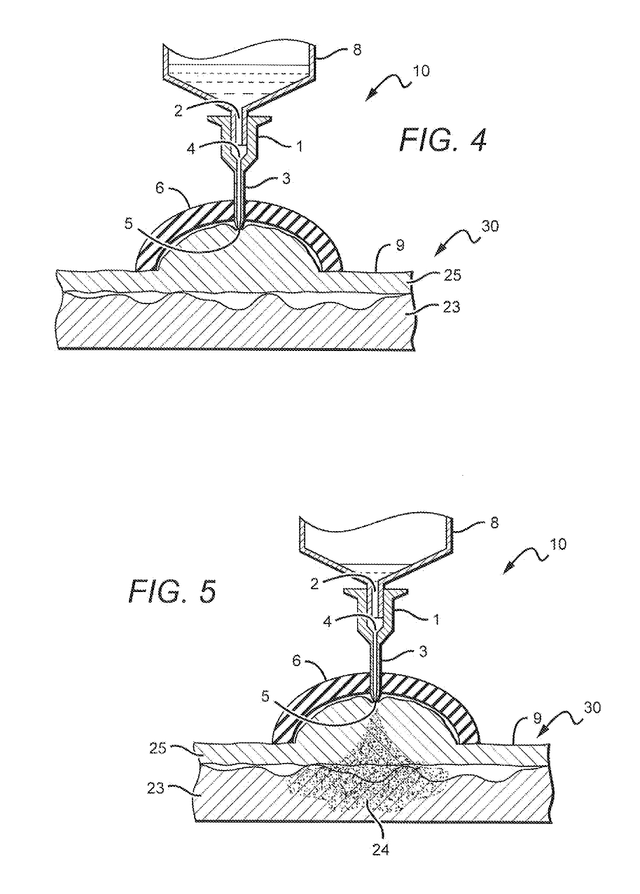

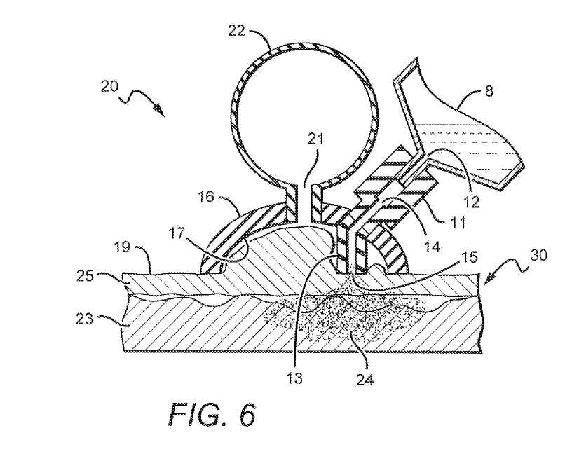

In one embodiment, as illustrated in FIGS. 3-5, the process or method of injection using the device illustrated in FIG. 1 can be as follows: a syringe 8 can be filled with a fluid such as a particular dosage of medication. The syringe 8 can then be attached to the hub 1 of the device by inserting syringe tip 12 into the hub 1. The integral unit, (the device 10 and the syringe 8), loaded with the selected dosage, can be grasped in the hand of a user (or any other suitable holding mechanism instead of a user), and held proximate to the epidermis in order to manually or mechanically inject the selected dosage into, through or under the epidermis. A negative pressure can then be created, for example, under the suction cup 6. In a device having a soft rubber cup 6, this could be achieved by pressuring the whole unit against the epidermis, thereby pushing the air out from under the suction cup. The negative pressure thus created in the suction cup 6 can create a negative pressure also in the epidermis 25 and the underlying tissue 23 or tissues, drawing the epidermis and underlying tissue to the injection port 5 where it can be pierced by that port 5. The user can then push down upon the plunger in the syringe 8, thereby driving the piston to eject the selected dosage of medication through the narrow orifice injection port 5 and into or under the epidermis 9.

### EXAMPLE 5

In one embodiment, #the process or method of injection using the device illustrated in FIG. 2 can be as follows: a syringe 8 can be filled with a fluid such as a particular dosage of medication. The syringe 8 can then be attached to the hub 11 of the device by inserting syringe tip 12 into the hub 1. The integral unit (the device 20 and the syringe 8), loaded with the selected dosage, can be grasped in the hand of a user (or any other suitable holding mechanism instead of a user), and held proximate to the epidermis 25 in order to manually or mechanically inject the selected dosage through, into or under the epidermis. The negative pressure can be created, for example, under a rigid or semi-rigid suction cup 16 by depressing the bulb 22 to empty the air out of it, positioning the integral unit against the surface 9 to be injected, and then releasing the bulb in order to draw the air out of the empty space and into the bulb. The negative pressure thus created in the suction cup 6 can create a negative pressure also in the epidermis 9 and the tissue, drawing the epidermis 9 or underlying tissue(s) 23, such as dermis and subcontaneous tissue, to the injection port 15 where it can be pierced by the injection port 15. The user can then push down upon the plunger in the syringe 8, driving the piston down to eject the selected dosage of medication 24 through the narrow orifice injection port and into or under the epidermis.

Alternatively, in a device having a soft rubber cup, a negative pressure or vacuum could be achieved by pressuring the whole unit against the epidermis, thereby pushing the air out from under the suction cup.

Referring to FIGS. 1 and 3-5, a device 10 according to the invention may be used for many suitable applications, including without limitation aspiration of fluids from a tissue such as blood sample aspiration for laboratory analysis. The device 10 may include a support member 1 having an opening for a reservoir tip 2, a channel member 3 having a first input port 4 and an injection port 5, wherein the injection port 5 comprises a small protrusion effective to provide injection or aspiration. The device 10 further may include a contact member 6 that surrounds the channel member 3, such that the injection port 5 in this embodiment can open into the contact member 6 and the channel member 3 has a through channel that fluidly connects the first input port 4 and the injection port 5. In this embodiment, the contact member 6 is positioned between the support member 1 and the injection port 5. The contact member includes an empty space 7, which is defined by the size, shape, or dimensions of the overlying contact member 6, and can be quickly, easily and accurately placed adjacent to a surface 9 to be injected or aspirated, such as epidermis. In this type of case, after applying the device 10 to the epidermis 9, the aspiration could made in any suitable manner, including without limitation by moving the syringe plunger in the opposite direction, or by attaching a vial with negative pressure inside.

Any suitable reservoir 8 such as any suitable fluid container can be used with the device, including without limitation the barrel and piston (plunger) of a typical disposable or reusable syringe.

The holder or support member 1 may be fabricated from any suitable material including without limitation plastic, glass and ceramics, metal, and combinations thereof. Any suitable size, shape or dimensions of the holder or support member 1 can be used, including for example, a holder in which one end is shaped like a syringe tip where a Luer lock or Luer-Slip fitting is suitable.

The injection port 5, such as a narrow injection orifice may have many suitable shapes and dimensions, including without limitation a small protrusion that facilitates the injection an opening having the form of a fissure that may, for example make the fluid jet plane as a knife, thus pushing the epidermis cells apart rather than puncturing. In one embodiment, the protrusion may be the size of a micro-needle. In another embodiment, the length of the protrusion may be less than or equal to 6 mm. In yet another embodiment, the length of the protrusion may be less than or equal to 3 mm. In a further embodiment, the length of the protrusion may be less than or equal to 1 mm The narrow orifice, which can open into the suction cup, may have a small surrounding protrusion that, after vacuum creation under the cup, facilitates the epidermis penetration by the fluid jet.

The contact member 6 may be a suction cup, which may be made of any suitable material, including without limitation soft rubber, silicone or plastic. The contact member 6 or suction cup may also have any suitable shape or form, including without limitation a concave or convex form. Furthermore, any suitable wall thickness can be used in order to create better a vacuum and/or more negative pressure in the tissue. The contact member 6 or suction cup may also include one or more additional structures or devices that allows or enhance the creation of negative pressure. In one embodiment, the contact member 6 or suction cup may be covered with a material that facilitates attachment of the device to the epidermis, including without limitation a gel or lotion having bactericidal or other capacities. It will be understood that while contact member 6 is shown as a suction cup, other suitable devices may be utilized to accomplish the same function.

The needle-free injection device 10 can be used for the administration of any type of fluid 24 including without limitation therapeutic medications. The device 10 can be attached to any reservoir 8, such as any suitable container, including without limitation a regular syringe. In one embodiment, the device 10 can replace a regular hypodermic needle.

The injection device 10 can be attached to any suitable container to form an injection system/unit, including without limitation a regular syringe (disposable or otherwise) filled with the medication already taken into the syringe by a regular method of fluid aspiration from a drug vial or other type of suitable container, including without limitation fluid aspirated through a needle. The device can be used to administer medications in any suitable manner, including without limitation intramuscularly, subcutaneously, or intracutaneously.

Any suitable container or device for delivering the fluid may be used for injections, including without limitation a syringe, a needle-free syringe, an ampule or the like.

Any suitable means of creating a negative pressure or vacuum may be used, including without limitation a suction cup, a bulb, a reverse piston or combinations thereof. Additionally, any other means of substantially bringing the epidermis of a subject or article in contact with the injection hole could be used.

Any suitable fluid, suspension or emulsion can be used with the invention, including without limitation a medication, supplement or electrolyte. In addition, any suitable support member 1 can be used including without limitation a hub into which a syringe tip can fit. Similarly, any suitable channel member can be used, including a duct comprising a fluid input port and distinct injection port fluidly connected by a through channel. The size, type or dimensions of the input port and the injection port can also vary according to the needs of the particular application desired, including without limitation the use of a narrow orifice having a short, sharp protrusion. Furthermore, any suitable contact member can be used including without limitation a resilient or flexible suction cup or a relatively non-flexible cup or cap. In addition, the size, shape and dimensions of the contact member can define the underlying empty space and can vary according to the particular application of use. The contact member may be fabricated from flexible or otherwise resilient materials in some applications, including without limitation plastic and rubber, and in relatively less flexible to rigid materials for other applications, including without limitation rigid or semi-rigid plastics. Any suitable negative pressure mechanism can be used, including without limitation a flexible or resilient bulb or a reverse piston.

While several illustrative embodiments of the invention have been disclosed herein, still further variations and alternative embodiments will occur to those skilled in the art. Therefore, the fluid injection apparatus forming one aspect of the invention can be useful wherever the distribution of fluids, emulsions or suspensions to a surface or subsurface is required and accordingly is amenable to a broad range of applications besides those described above, including without limitation veterinary and food processing application. Finally, the shape and dimensions of the systems, devices and their components can vary depending upon the particular application, including without limitation injection into different thicknesses of skin or different regions of a body. Such variations and alternative embodiments are contemplated, and can be made without departing from the spirit and scope of the invention as defined in the appended claims.

We claim:

1. A needle-free injection or aspiration device comprising:
a support member comprising a first opening for a reservoir;
a channel member comprising a first input port and an injection port, wherein the first input port is fluidly connected to the support member and the injection port comprises a small protrusion effective to provide injection or aspiration;
a contact member surrounding at least one portion of the channel member, wherein the contact member is positioned between the support member and the injection port; and
an empty space underlying the contact member,
wherein the size, shape or dimensions of the contact member are effective to define the underlying empty space.

2. The device of claim 1, wherein the injection port comprises a narrow orifice.

3. The system of claim 1, wherein the small protrusion is a micro-needle.

4. The device of claim 3, wherein the length of the protrusion is less than or equal to 6 mm.

5. The device of claim 3, wherein the length of the protrusion is less than or equal to 3 mm.

6. The device of claim 1, wherein the contact member is flexible.

7. The device of claim 1, further comprising a reservoir wherein the reservoir is fluidly connected to the support member.

8. The device of claim 7, wherein the reservoir is a needle-free syringe.

9. The device of claim 1, wherein the injection device is at least one of hypodermic, transdermal, intramuscular, subcutaneous and intracutaneous.

10. The device of claim 1, wherein the device is disposable.

11. The device of claim 1, further comprising a negative pressure mechanism effective to generate a negative pressure in the empty space when the contact member is adjacent to a surface, wherein the contact member comprises a second input port and the negative pressure mechanism is physically connected to the second input port.

12. The device of claim 11, wherein the negative pressure is effective to position at least one portion of the surface adjacent to the small protrusion in the injection port, thereby providing penetration of the at least one portion of the surface.

13. The device of claim 11, wherein the negative pressure mechanism is one of a resilient bulb or a reverse piston.

14. The device of claim 1, wherein the reservoir is a needle-free syringe.

15. A needle-free injection or aspiration system comprising:
a support member comprising a first opening for a reservoir;
a channel member comprising a first input port and an injection port, wherein the first input port is fluidly connected to the support member and the injection port comprises a small protrusion effective to provide injection or aspiration;
a contact member surrounding at least one portion of the channel member, wherein the contact member is positioned between the support member and the injection port;
an empty space underlying the contact member, wherein the size, shape or dimensions of the contact member are effective to define the underlying empty space;
a reservoir, wherein the reservoir is fluidly connected to the support member;
wherein the contact member further comprises a second input port;
a surface adjacent to the empty space; and
a negative pressure mechanism connected to the second input port on the contact member, wherein the mechanism is effective to generate a negative pressure in the empty space between the contact member and the surface,
wherein the negative pressure or vacuum is effective to position at least one portion of the surface adjacent to the small protrusion in the injection port, and thereby penetrating the at least one portion of the surface.

16. The system of claim 15, wherein the device is disposable.

17. The system of claim 15, wherein the surface is one of a natural or a synthetic outer layer of tissue that covers at least one portion of the body of a person or animal.

18. The system of claim 15, wherein the device is disposable.

19. An injection method of using a needle-free injection or aspiration device having a flexible contact member, the method comprising:
filling a reservoir with a selected dosage of fluid;
attaching the reservoir to the first opening in the support member, thereby forming a fluid connection between the reservoir and the support member;
positioning the contact member adjacent to a surface;
pressing down on the device in a manner effective to create a negative pressure between at least one portion on the surface and the contact member, thereby penetrating the at least one portion of the surface underlying the contact member; and
increasing the pressure in the reservoir, thereby injecting the selected dosage into the at least one portion of the surface.

20. The method of claim 19, wherein the surface comprises the epidermis of a person or animal.

21. An injection method of using a needle-free injection or aspiration device having a negative pressure mechanism, the method comprising:
filling a reservoir with a selected dosage of fluid;
attaching the reservoir to the first opening in the support member, thereby forming a fluid connection between the reservoir and the support member;
positioning the contact member adjacent to a surface;
activating the negative pressure mechanism in a manner effective to create a negative pressure between at least one portion of the surface and the contact member, thereby penetrating the at least one portion of the surface underlying the contact member; and
increasing the pressure or decreasing the volume in the reservoir, thereby injecting the selected dosage into the at least one portion of the surface.

22. The method of claim 21, wherein the surface comprises the epidermis of a person or animal.

23. An aspiration method of using a needle-free injection or aspiration device having a flexible contact member, the method comprising:
providing an empty reservoir;
attaching the reservoir to the first opening in the support member, thereby forming a fluid connection between the reservoir and the support member;
positioning the contact member adjacent to a surface;
pressing down on the device in a manner effective to create a negative pressure between at least one portion of the surface and the contact member, thereby penetrating the at least one portion of the surface underlying the contact member; and
decreasing the pressure within the reservoir, thereby aspirating fluid from within the surface and into the reservoir.

24. The method of claim 23, wherein the surface comprises the epidermis of a person or animal.

25. An aspiration method of using a needle-free injection or aspiration device having a negative pressure mechanism, the method comprising:
providing an empty reservoir;
attaching the reservoir to the first opening in the support member, thereby forming a fluid connection between the reservoir and the support member;
positioning the contact member adjacent to a surface;
activating the negative pressure mechanism in a manner effective to create a negative pressure or vacuum between at least one portion of the surface and the contact member, thereby penetrating the at least one portion of the surface underlying the contact member; and
decreasing the pressure within the reservoir, thereby aspirating fluid from within the at least one portion of the surface and into the reservoir.

26. The method of claim 25, wherein the surface comprises the epidermis of a person or animal.
